(19) United States
(12) Patent Application Publication
(10) Pub. No.: US 2025/0058082 A1
(43) Pub. Date: Feb. 20, 2025

(54) TRANSPERINEAL, TRANSRECTAL OR TRANSURETHRAL ADMINISTRATION OF ANTI-PROSTATE CANCER AGENTS FOR TREATING PROSTATE CANCER

(71) Applicant: JESSENIUS FACULTY OF MEDICINE OF COMENIUS UNIVERSITY IN MARTIN, Martin (SK)

(72) Inventors: Jan SVIHRA, Martin (SK); Juraj MIKLUSICA, Martin (SK); Peter ZVARA, Odense M (DK); Eduard BACO, Oslo (NO)

(73) Assignee: JESSENIUS FACULTY OF MEDICINE OF COMENIUS UNIVERSITY IN MARTIN, Martin (SK)

(21) Appl. No.: 18/720,189

(22) PCT Filed: Dec. 13, 2022
(86) PCT No.: PCT/EP2022/085483

§ 371 Date: Jun. 14, 2024

(30) Foreign Application Priority Data
Dec. 14, 2021 (EP) 21214512.2

(51) Int. Cl. A61M 25/00; A61K 9/00; A61K 31/045

(52) U.S. Cl. CPC A61M 25/0084; A61K 9/0034; A61K 31/045; A61M 2025/0089

(57) ABSTRACT

The present invention relates to compositions comprising an anti-prostate cancer agent for use in a method of focally treating prostate cancer, as well as methods of focally treating prostate cancer using such anti-prostate cancer agents.

### CROSS-REFERENCE TO RELATED APPLICATION

The present application claims the benefit of priority of EP patent application Ser. No. 21/214,512.2 filed on 14 Dec. 2021, the content of which is hereby incorporated by reference in its entirety for all purposes.

The present invention relates to compositions comprising an anti-prostate cancer agent for use in a method of focally treating prostate cancer, as well as methods of focally treating prostate cancer using such anti-prostate cancer agents.

Prostate cancer (PCa) is the second most common cancer in men worldwide. 1.3 million new cases were diagnosed in 2017, a 42% increase from 2007. Introduction of prostatic specific antigen (PSA) screening resulted in more than 90% of PCa being diagnosed while still localized to the prostate (Global Burden of Disease; Cancer C et al., JAMA Oncol (2019)).

Many oncologic strategies exist for tissue-preserving treatment of other organs, however standard-of-care for PCa treatment remains focused on treatment of whole organ with surgery or radiation. Both radical prostatectomy and whole gland radiation are invasive, which often makes them non-viable for use in patients with co-morbidities. In younger, low-risk patients, the survival benefits of these therapies are often contrasted with the debilitating life-long side effects that develop due to damage to the periprostatic structures, such as the urinary sphincter, neurovascular bundle, bowel and bladder. The most frequent complications of radical prostatectomy include permanent urinary incontinence necessitating pad use in 17-54% and erectile dysfunction in around 80% of patients. Rectal toxicity associated with rectal bleeding, fecal incontinence and pain, develops in over 20% of patients receiving radiation therapy (Holm et al., Scand J Urol (2015), 49 (3): 250-259; Østby-Deglum et al., Scan J Urol (2015), 49 (6): 433-439; Donovan et al., New Engl J Med (2016), 375 (15): 1425-1437; Resnick et al., N Engl J Med (2013), 368 (5): 436-445).

In recent years, local therapy based on heat and freezing of the prostate tissue have been studied such as high intensity focused ultrasound, photodynamic therapy, electroporation and cryotherapy. However, these therapies are dependent on complex technologies with complication rates varying greatly among centers. None were introduced into broad clinical practice. The complications, while lower than with surgery and radiation, are still frequent because the heat affects adjacent tissue, most notably nerves controlling erection or muscles controlling urinary continence.

It was previously documented that prostatic pseudocapsule (fibromuscular sheath encircling the prostate) stops the diffusion of the medicament in fluid, injected into the prostate, into the periprostatic area (Plante et al., NJU International (2004), 94 (9): 1384-1388). This is an advantage compared to heat. The prostate however contains several tissue types (glandular tissue, smooth muscle, and fibrous stroma). When classic hollow core needle with a single point of injection at the needle tip is used, these tissue types display varying degree of resistance, causing variable degrees of intraprostatic diffusion and backflow of the injected substance along the needle tract (Plante et al., BJU International (2003), 91 (1): 94-98; Plante et al., BJU International (2004), 94 (9): 1384-1388).

Recent research has shown that while PCa is often multifocal, the disease is likely driven by the largest and highest grade index lesion, which can be reliably identified in 80% of cases by multiparametric magnetic resonance imaging (mpMRI) (Baco et al., Eur Urol (2016), 69 (1): 149-156; Ahmed et al., N Eng J Med (2009), 361 (17): 1704-0706; Baco et al., Eur Urol (2015), 67 (4): 787-794). As a result of the increasing number of diagnoses with localized involvement, the number of patients receiving radical prostatectomy and whole gland radiation therapy is increasing, which is associated with significant genitourinary and rectal side effects (Welch et al., J Natl Cancer Inst (2009), 101 (19): 1325-1329; Donovan et al., N Engl J Med (2016), 375 (15): 1425-1437). Because of these factors, focal therapies that target the clinically significant PCa lesion(s) while preserving healthy prostate tissue and adjacent anatomical structures are increasingly needed (Valerio et al., Eur Urol (2014), 66 (4): 732-751). Attempts to develop aforementioned techniques have been limited by inability to safely distribute injectant and localize injection location (Tammela et al., J Urol (2017), 198 (6): 1333-1339; Grise et al., Eur Urol (2004), 46 (4): 296-501, discussion 501-492).

The prostatic pseudocapsule, a 2-3 mm thick fibromuscular layer surrounding the prostate and separating it from periprostatic structures, acts as a barrier to the diffusion of injected 96% ethanol (EtOH), thus presenting an anatomic advantage over temperature-based focal treatment options, like cryotherapy or high-intensity focused ultrasound (Zvara et al., Urology (1999), 54 (3): 411-415; Plante et al., BJU International (2004), 94 (9): 1384-1388). In addition, significant improvement in MRI and MRI-transrectal ultrasound-MRI-TRUS fusion have aided in accurately identifying clinically significant prostate lesions for biopsy (Baco et al., Eur Urol (2015), 67 (4): 787-794; Curnud et al., Abdom Imaging (2013), 38 (6): 1447-1463; Delongchamps et al., J Urol (2015), 193 (4): 1198-1204).

Given the functional advantage of the prostatic pseudocapsule and improved imaging ability, the need for a complementary injection technique is critical to the development of novel PCa therapies and drug delivery methods.

These and further disadvantages need to be overcome. The present invention therefore addresses these needs and technical objectives and provides a solution as described herein and as defined in the claims.

The present invention relates to a composition comprising an anti-prostate cancer agent for use in a method of focally treating prostate cancer in a human patient, said method comprising the step of transperineally, transrectally, or transurethrally administering through a hollow fiber membrane portion of a catheter a pre-determined volume of said composition which pervades essentially (e.g., at least 80, 90, 95, or 99%, or 100% of) the entire volume of a pre-determined intraprostatic lesion.

In context with the present invention, it was shown that intraprostatic injection of EtOH causes coagulative necrosis and tissue ablation. Further, in contrast to a standard needle with a single opening at its tip, injection efficacy and injectant distribution as applied in accordance with the present invention was significantly improved with the use of a hollow fiber membrane catheter, allowing diffusion of EtOH along the entirety of the segment. The findings provide a strong foundation for further exploration of the porous needle as a component of modern PCa therapy protocols.

Also, as has been demonstrated herein in accordance with the present invention, focal treatment of prostate cancer is ideal for treatment without destroying non-lesional prostate tissue thus preserving healthy prostate tissue and adjacent anatomical structures. In one embodiment of the present invention, the focal treatment is conducted in a way that the lesion is fully (i.e. at least 80, 90, 95, or 99%, or 100%) filled up or saturated with the anti-prostate cancer agent. The focal treatment as to be employed in accordance with the present invention is in contrast to the treatment of benign prostate hypertrophy (BPH) where prostate tissue is debulked since BPH merely obstructs the urethra and causes impairment of bladder emptying and therefore debulking of the prostate is needed, irrespective of which tissue is destroyed or excised (which treatment is thus non-focal). PCa however requires focal ablation (eradication) of the lesions to halt the disease progression into disseminated (metastatic) disease. This refers to the most crucial difference between both BPH and PCa treatments. Also, BPH does not cause such lesions like in prostate cancer (PCa), and is in the center of the prostate, whereas PCa is in the periphery. Particularly, in such PCa treatment which differs from the BPH treatment mentioned above, the pre-determined volume of said composition is based on a linear correlation between the to be administered volume of said composition and the volume of said predetermined lesion. Svihra et al., J Urol Ren Dis (2020), 5 (4): 1-8 describes such non-focal treatment of BPH by administering EtOH using a microporous needle via transurethral intraprostatic injection to a patient. In the context of treating BPH, TRUS-guided transrectal and TRUS-transperineal intraprostatic injections have also been mentioned by Svihra et al. (2020), where it was disclosed that adverse events occurred. King et al., Journal of Urology (2012), 187 (5): 1898-1902 as another prior art document focuses again on a potential treatment of prostate disease such as BPH by applying transrectally intraprostatic injection of EtOH via a TRUS guided microporous hollow fiber catheter (MiHFC). The study was applied to dogs. Transurethral intraprostatic injection has also been mentioned as another route of administration in King et al. (2012). A follow up study of King et al. (2012) (see King et al., Prostate Cancer and Prostatic Disease (2015), 18:237-241) discloses an experimental model in humans with regard to a BPH treatment. In detail, the application of EtOH via only transrectal intraprostatic injection with a TRUS guided MiHFC is mentioned. Again, the occurrence of adverse events have been stated for a intraprostatic transperineal injection when treating a prostate disease such as BPH. All of these prior art documents however refer to the treatment of BPH which is based on the abovementioned something completely different as the treatment of PCa in a subject in need thereof. Mutaguchi et al., Urology (2006), 68:307-311 also focuses on a transurethral EtOH injection via an indwelling catheter into the prostate of patients with persistent urinary retention resulting from BPH or advanced prostate cancer. This study however does not apply a catheter with a hollow fiber membrane portion according to the invention. Transperineal or transrectal intraprostatic injection has also not been mentioned.

In accordance with the present invention, focal treatment is achieved by the hollow fiber membrane catheter as defined in the claims and also further described and exemplified herein.

In context with the present invention, in order to support administering the anti-prostate cancer agent to the ideal spot, it may be beneficial to precisely guide the hollow fiber membrane portion of the catheter as described herein toward the prostate for the treatment of PCa. In accordance with the present invention, this step may help to improve the accuracy of the catheter placement and provide effective diffusion of the drug within the PCa lesion for focal treatment while preserving surrounding healthy tissue. Accordingly, in one embodiment of the present invention, the step of transperineally, transrectally, or transurethrally administering through a hollow fiber membrane portion of a catheter a pre-determined volume of said composition which pervades essentially the entire volume of a pre-determined intraprostatic lesion is preceded by a step of transperineally, transrectally or transurethrally positioning the hollow fiber membrane portion of the catheter comprising said composition comprising an anti-prostate cancer agent in the geometrical center of a pre-determined intraprostatic lesion, such that the middle of said hollow fiber membrane portion is in the geometrical center of the intraprostatic lesion (cf. FIG. 3).

In a specific embodiment of the present invention, said positioning step where the hollow fiber membrane portion of the catheter is transperineally, transrectally or transurethrally positioned in the geometrical center of said pre-determined intraprostatic lesion prior to transperineally, transrectally, or transurethrally administering through a hollow fiber membrane portion of a catheter a pre-determined volume of said composition comprising an anti-prostate cancer agent is conducted using magnetic resonance imaging (MRI), e.g., MP (multiparametric)-MRI or MRI-TRUS (transrectal ultrasound), particularly MRI-TRUS. Improvements to multiparametric MRI allow for precise localization of PCa lesions within the prostate. In accordance with the present invention and as shown herein, the ability to target the lesion for biopsy may improve under MRI-TRUS fusion, an imaging method using a 3D MRI image of the prostate, taken beforehand, which is subsequently fused with a real-time TRUS image (Cornud et al., Abdom Imaging (2013), 38 (6): 1447-1463). Combined with the effective injection technique as provided in context with the present invention, this procedure which is routinely used to take prostate biopsies, may be used for targeting the PCa lesion for drug delivery as described herein. Methods for MRI-TRUS are known in the art and also described, e.g., in Cool et al., Can Urol Assoc J (2016), 10 (9-10): 342-348 or U.S. Pat. No. 8,702,579.

In accordance with the present invention, the volume of the anti-prostate cancer agent to be administered as described herein may be correlated with the volume of the (pre-determined) lesion where agent is administered to. By appropriately correlating the volume of the anti-prostate cancer agent to be administered as described herein with the volume of the (pre-determined) lesion where agent is administered to, both effective treatment of PCa and decreasing or avoiding the generation of necrosis in the area surrounding PCa can be achieved. Accordingly, in one embodiment of the present invention, the pre-determined volume of said composition comprising the anti-prostate cancer agent may be based on a linear correlation between the to be administered volume of said composition and the volume of said (pre-determined) lesion. In one embodiment of the present invention, both the position of the lesion as well as its volume are pre-determined prior to transperineal, transrectal, or transurethral administration of said composition comprising the anti-prostate cancer agent to the intraprostatic lesion as described in context with the present invention. For example, in accordance with the present invention, both the positioning and the volume determination of the lesion may be conducted using magnetic resonance imaging (MRI), e.g., MP (multiparametric)-MRI or MRI-TRUS (transrectal ultrasound), particularly MRI-TRUS.

In a specific embodiment of the present invention, the linear correlation between the to be pre-determined administered volume of said composition and the volume of said (pre-determined) lesion may be determined by the following formula:

$$y = {0.7634}^{*} x - 0.5488 \qquad ( Formula I )$$

wherein y corresponds to the volume of said predetermined lesion, in ml, and x corresponds to the volume of said to be administered composition, in ml; with a regression analysis of R² being 0.4812.

In another specific embodiment of the present invention, the pre-determined volume of said composition comprising the anti-prostate cancer agent be administered to the intraprostatic lesion as described in context with the present invention may be calculated by the following formula:

$$x = ( y + 0.5488 ) / 0.7634 \qquad ( Formula II )$$

wherein y corresponds to the volume of said predetermined lesion, in ml, and x corresponds to the pre-determined volume of said composition to be administered, in ml.

In accordance with the present invention, the intraprostatic lesion where the composition comprising the anti-prostate cancer agent is transperineally, transrectally, or transurethrally administered to through a hollow fiber membrane portion of a catheter, is pervaded with said composition within a reasonable amount of time. In one embodiment of the present invention, said composition pervades essentially (e.g., at least 80, 90, 95, or 99%, or 100% of) the entire volume of the pre-determined intraprostatic lesion, e.g. within 1 to 4 hours, preferably 1 to 3 hours, more preferably 1 to 2 hours after administration.

In accordance with the present invention, the composition comprising the anti-prostate cancer agent may be transperineally, transrectally, or transurethrally administered through a hollow fiber membrane portion of a catheter to the pre-determined intraprostatic lesion as described herein by any suitable means. In one embodiment of the present invention, said composition is administered by the aid of a pump, e.g., an infusion pump. In a specific embodiment of the present invention, the composition comprising the anti-prostate cancer agent is administered to the lesion at a rate of about 10-20, preferably about 15 ml per hour.

As described and provided herein in accordance with the present invention, the composition comprising the anti-prostate cancer agent is transperineally, transrectally, or transurethrally administered to the pre-determined intraprostatic lesion through a hollow fiber membrane portion of a catheter. In accordance with the present invention, said hollow fiber membrane of said catheter may have any shape or characteristic which allows proper distribution of said composition to the lesion as described herein. In a specific embodiment of the present invention, said hollow fiber membrane of said catheter may have a wall thickness of 100+/−about 25 μm, and/or an inner diameter of 300+/−about 40 μm. In a further specific embodiment of the present invention, said hollow fiber membrane of said catheter may have the following performance characteristics: a maximum pore size of 0.5+/−about 0.1 μm, and/or a transmembrane flow (H₂O, 25° C.)≥35 ml/[min×cm²×bar]. In a further specific embodiment, said hollow fiber membrane of said catheter may have a length of about 0.5 to about 5 cm, preferably about 1 to 3 cm. In a further specific embodiment of the present invention, said hollow fiber membrane of said catheter may be made of polyethersulfone. A particular specific example for a hollow fiber membrane of said catheter which can be employed in accordance with the present invention is MicroPES® capillary membrane Type TF10 (3M®, Charlotte, NC, USA), e.g., as described in Sheet No. 433/0091/000 of March 2016.

Furthermore, in accordance with the present invention, said catheter with the hollow fiber membrane which is used to administer said composition comprising the anti-prostate cancer agent to the intraprostatic lesion may be comprised by a catheter deployment system.

Generally, in accordance with the present invention, any anti-prostate cancer agent may be used which can be administered to an intraprostatic lesion using a hollow fiber membrane of a catheter. As apparent for those skilled in the art, such anti-prostate cancer agent must be fluid or solved in a fluid to allow proper passing of the agent through hollow fiber membrane into the lesion. Suitable examples of such anti-prostate cancer agents which can be used in accordance with the present invention comprise inter alia non-steroidal antiandrogen (NSAA) agents such as 2-hydroxyflutamide, or ethanol (C₂H₅OH).

The present invention further relates to a method of focally treating prostate cancer in a human patient, said method comprising transperineally, transrectally, or transurethrally administering a pre-determined volume of a composition comprising an anti-prostate cancer agent through a hollow fiber membrane of a catheter to said patient in need thereof, wherein said composition pervades essentially (e.g., at least 80, 90, 95, or 99%, or 100% of) the entire volume of a pre-determined intraprostatic lesion.

Also, the present invention relates to the use of an anti-prostate cancer agent for the preparation of a medicament for the treatment of prostate cancer in a patient, wherein a pre-determined volume of said medicament is to be administered transperineally, transrectally, or transurethrally through a hollow fiber membrane of a catheter to said patient, wherein said composition pervades essentially (e.g., at least 80, 90, 95, or 99%, or 100% of) the entire volume of a pre-determined intraprostatic lesion.

The embodiments which characterize the present invention are described herein, shown in the Figures, illustrated in the Examples, and reflected in the claims.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% or 2% of a given value or range.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

All publications and patents cited throughout the text of this specification (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

### FIGURES

The Figures show:

FIG. 1 Volume of tissue lesions in all canine prostates. The volume injected is listed in brackets under x axis, the volume of the lesion is above each bar. Dog 1 represents a dose finding experiment and the asterisk represents that a 2 cm-long porous needle was used in Dog 4.

FIG. 2 Correlation of the volume of ethanol and the volume of necrotic lesion in all canine experiments where 1 cm long porous segment was used for injection. Regression analysis demonstrating strong correlation R² of 0.85.

FIG. 3 Schematic figure of positioning the hollow fiber membrane portion of the catheter comprising said composition comprising an anti-prostate cancer agent in the geometrical center of a pre-determined intraprostatic lesion, such that the middle of said hollow fiber membrane portion is in the geometrical center of the intraprostatic lesion

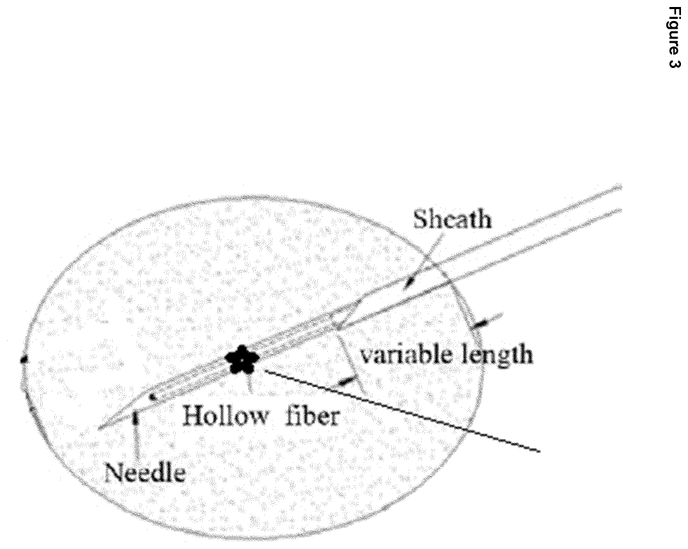

FIG. 4 Necrotic areas in the canine prostate, involving more than 80% of the section and extending to the prostatic pseudocapsule, without causing any disruption.

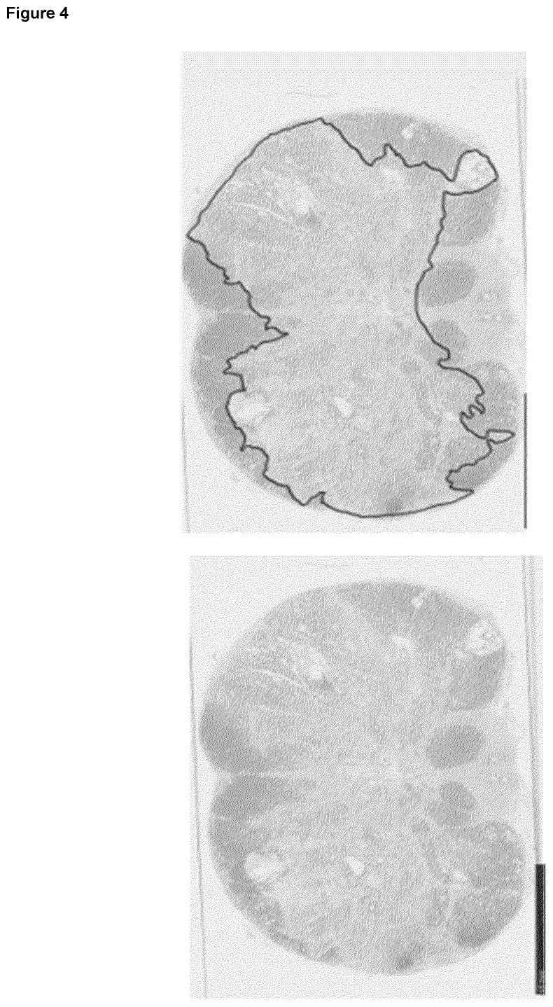

FIG. 5 Whole mount sections of the prostate of a dog showing gross tissue sections (left) and corresponding hematoxylin and eosin-stained 5 μm sections (right) demonstrating necrotic lesions following intraprostatic injection of 1 and 2 mL of EtOH into each lobe

FIG. 6 High power magnification marked by arrow is depicting intact fibromuscular pseudocapsule of a canine prostate adjacent to the necrotic lesion.

FIG. 7 Tissue defects developed 4 weeks after intraprostatic injection of absolute ethanol. A: 2 ml (left) and 0.5 ml (right), B: 2 ml (left) and 1 ml (right), C: 1.5 mL on both sides. Tissue defects lined with epithelium and surrounded by connective tissue with signs of fibrosis. The area of the prostatic pseudocapsule with no abnormalities.

FIG. 8 Volume of tissues lesions in five human prostates. The volume injected is listed under the x axis, i.e., 2 ml or 3 ml, respectively. The average volume of the lesion is indicated inside the bar, i.e., 0.978 ml or 1.742 ml.

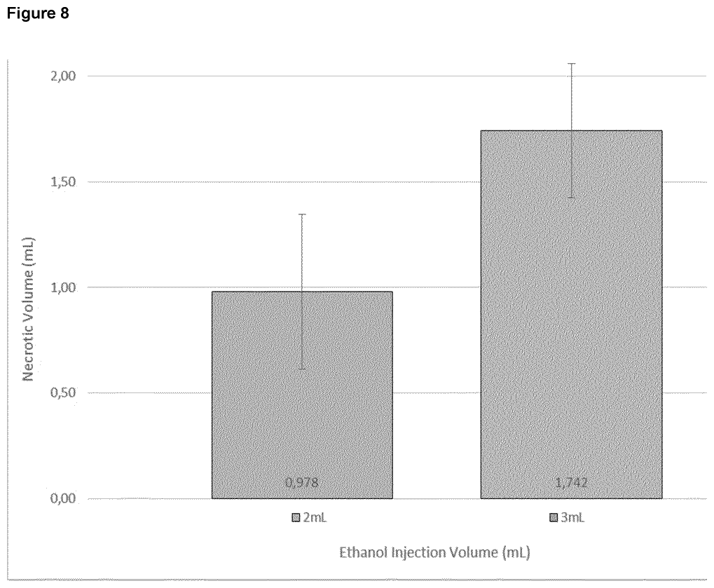

FIG. 9 Whole mount sections of the human prostate showing gross tissue sections (left) and corresponding hematoxylin and eosin-stained 5 μm sections (right) demonstrating necrotic lesions following intraprostatic injection of 2 and 3 mL of EtOH into left and right lobe respectively. Here, prostate lesions with irregularities in their shape are depicted to document the difference compared to dog.

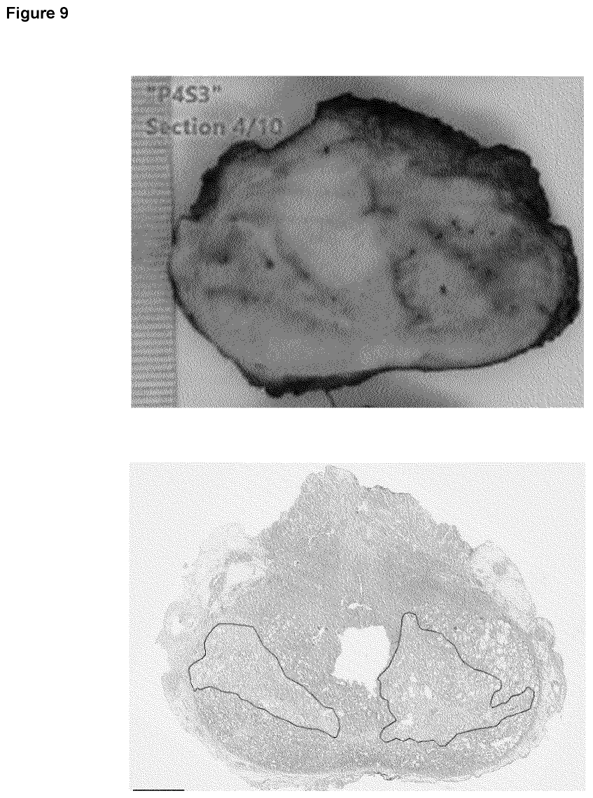

FIG. 10 Correlation of the volume of ethanol and the volume of necrotic lesion of five human prostates. Regression analysis demonstrating moderate correlation R² of 0.4812.

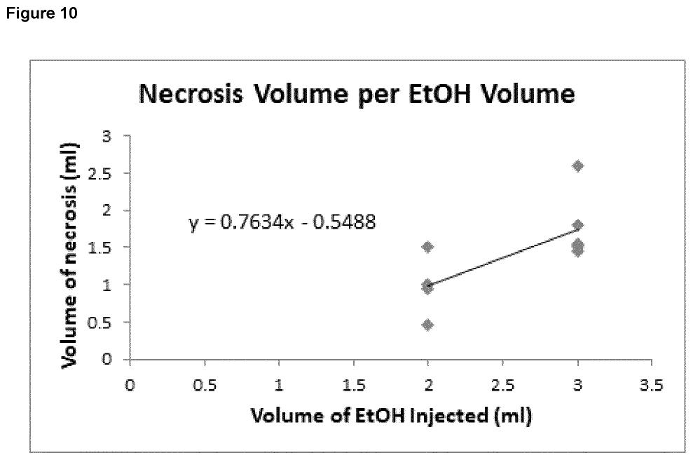

The present invention is further illustrated by the following examples. Yet, the examples and specific embodiments described therein must not be construed as limiting the invention to such specific embodiments.

### EXAMPLES

In acute experiments using a canine animal model, histological analysis was conducted two hours following intraprostatic EtOH injection with a porous needle. Lesion volumes were measured and analyzed for dose-dependency. In chronic studies, the safety of this procedure was assessed by closely observing the dogs over four weeks post-injection and the prostates and adjacent anatomical structures at the time of autopsy were evaluated. Histological analysis of the whole mount section of the prostate was used to address the long-term effects of EtOH.

### Acute Animal Experiment

Intraprostatic ethanol injections were performed in eight Beagle dogs for acute experiments. Animals received premedication with medetomidin (0.004 mg/kg), metadon (0.3 mg/kg), and acepromazil (0.02 mg/kg). Anesthesia was induced with propofol (1.5-2 mg/kg) and maintained by propofol (10 mg/kg/hour) and fentanyl (0.24 mg/kg/hour). Prostate exposure was performed using lower midline abdominal incisions and blunt dissection of surrounding tissues. The porous needle was stabilized with a fixation apparatus attached to the operating table and was deployed with a protective sheath in the lateral lobe of the prostate. Once the needle was in position, the porous segment was exposed by retracting the needle sheath. Where possible, the distal end of the sheath was kept at least 2 mm from the pseudocapsule. The EtOH was delivered using an infusion pump (Legato 210, KD Scientific, Holliston, MA, USA) at a rate of 15 ml/hour. The needle was left deployed in the prostate until the pressure returned to baseline. Injections in both lateral lobes of each canine were performed. In the dose finding experiment, 2 and 3 ml of EtOH were administered into the left and right lobe of the prostate, respectively, in order to determine the maximal possible dose. In the remaining experiments, 0.5, 1, 1.5, or 2 ml was injected into each prostatic lobe. In five experiments, a needle with a 1 cm long microporous segment was used, while a 2 cm long segment was used in one experiment. The tissue resistance during injections was measured by including the pressure transduced between the pump and the needle. Two hours following the second injection, the animals were anaesthetised by an intramuscular injection of medetomidine and ketamine, followed by intravenous injection of pentobarbital, and euthanized using exsanguination. Prostates were resected and periprostatic area was carefully inspected for signs of extraprostatic EtOH effects.

### Chronic Animal Experiment

Intraprostatic EtOH injections, using the same procedure as above, were performed in eight Beagle dogs for chronic experiments. 0.5, 1, 1.5, and 2 ml of EtOH were injected using same injection technique. The laparotomy was closed in two layers. The blood alcohol levels were measured in a sample taken from the jugular artery 20 minutes after the second intraprostatic EtOH injection. The animals were closely observed. During the first 24 hours post-op, animals were examined in 4-5-hour intervals for symptoms of pain, distress, fever, and problems with voiding. Daily observation was carried out throughout the postoperative period. Animals were weighed every 4 days. Any deviation from normal was recorded. The euthanasia followed by prostatectomy and inspection of periprostatic area was performed at four weeks after intraprostatic injection.

### Experiment Performed in Cadaveric Organ Donor

Prostates of hearth-beating cadaveric organ donors were injected with EtOH transperineally under the real time visualization with transrectal ultrasound. With the patient in the standard lithotomy position (lying on his back with legs placed in straps) the perineal skin was cleaned with betadine. Ultrasound probe was inserted into the rectum and secured in place using a probe holder. Subsequently the needle was guided into the prostate using a grid attached to the transrectal ultrasound probe. Porous section of the needle protected by a sheath was guided into the center of the lateral lobe of the prostate. Once the needle was in position, the porous segment was exposed by retracting the sheath. The EtOH was delivered using an infusion pump (Legato 210, KD Scientific, Holliston, MA, USA) at a rate of 15 ml/hour. The needle was left deployed in the prostate until the predetermined volume of EtOH was administered.

Injections in both lateral lobes were performed, administering either 2 or 3 ml of EtOH per prostate lobe. The prostate was harvested minimum of 6 hours following the second injection (6-16 hours).

### Tissue Processing and Histological Examination

The procedures used were described in detail in King et al., J Urol (2012), 187 (5): 1898-1902. Briefly, whole prostates were fixed in 10% neutral buffered formaldehyde, then sectioned into 2.5 mm step sections, which were oriented parallel to the base of the prostate. 5 μm thick whole mount sections were placed on glass slides and stained with hematoxylin and eosin. Histology slides were converted to digital images. Areas of necrosis were delineated, and sections were stacked to reconstruct lesions to determine volume with stereology software Stereo Investigators 9.14.5 (Microbrightfield Bioscience, Williston, Vermont). Five to seven sections per prostate were used to create a 3-dimensional (3D) image of the lesion.

### Statistical Methods

Five animals (total of 10 intraprostatic necrotic lesions), injected with 0.5-2.0 ml of EtOH through a 1 cm microporous segment, were included in the acute canine experiment statistical analysis. Five human prostates (total of 10 intraprostatic necrotic lesions), injected with either 2.0 or 3.0 ml of EtOH through a 1 cm microporous segment were also included. Calculated volumes were expressed as average±standard deviation and compared with a paired t-test. A regression analysis was used to identify relationships between the volume of ethanol injected and the size of the necrotic lesion.

### Results

### Acute Animal Study

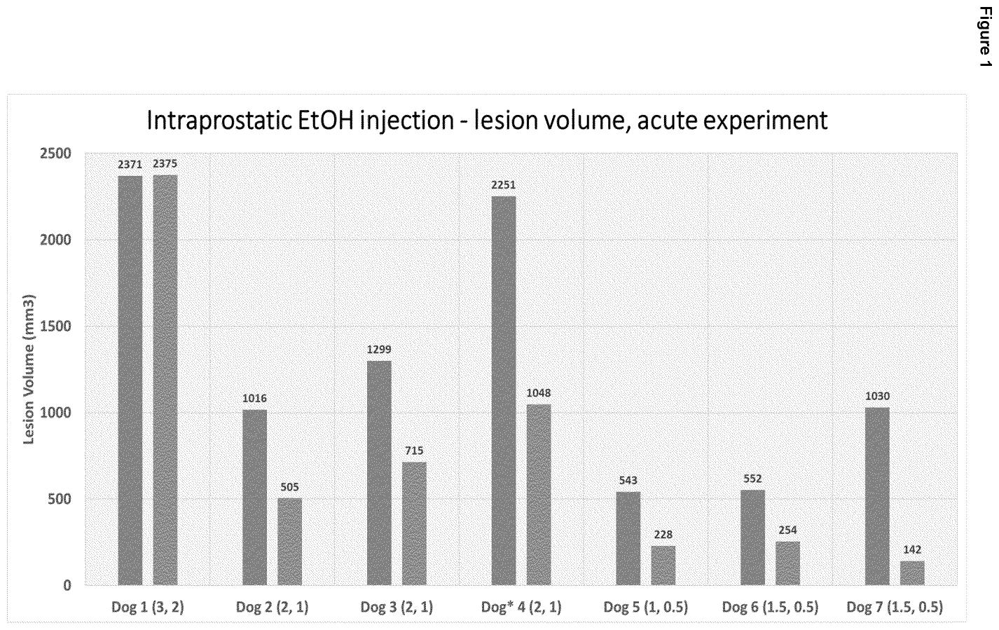

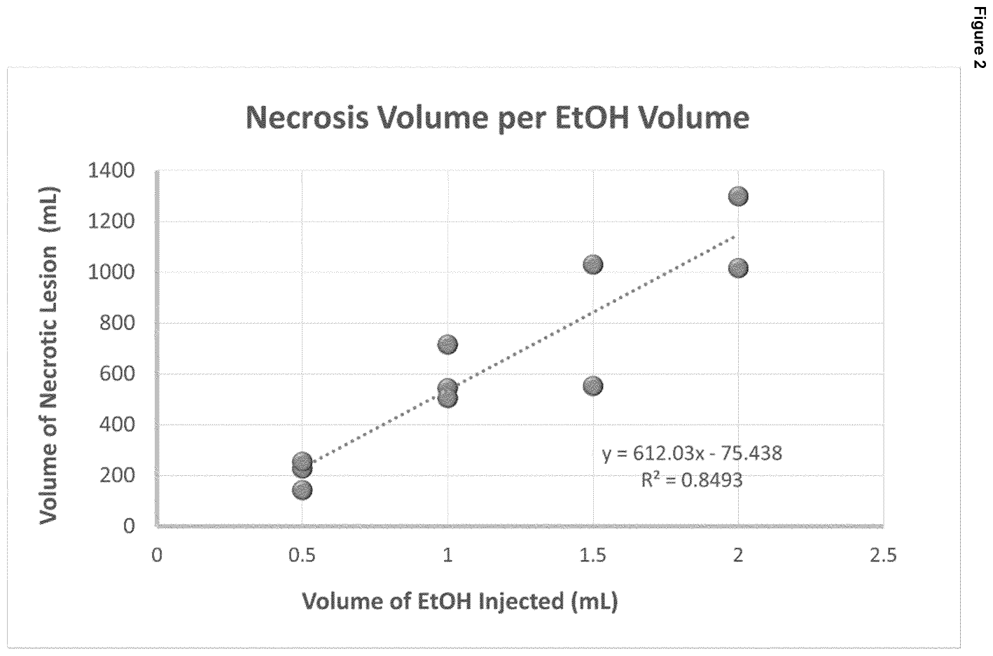

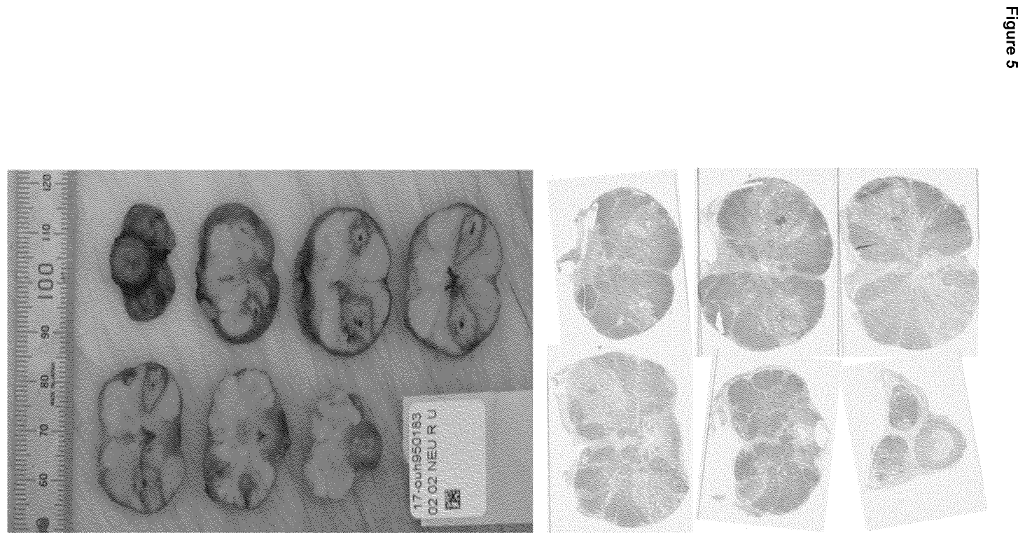

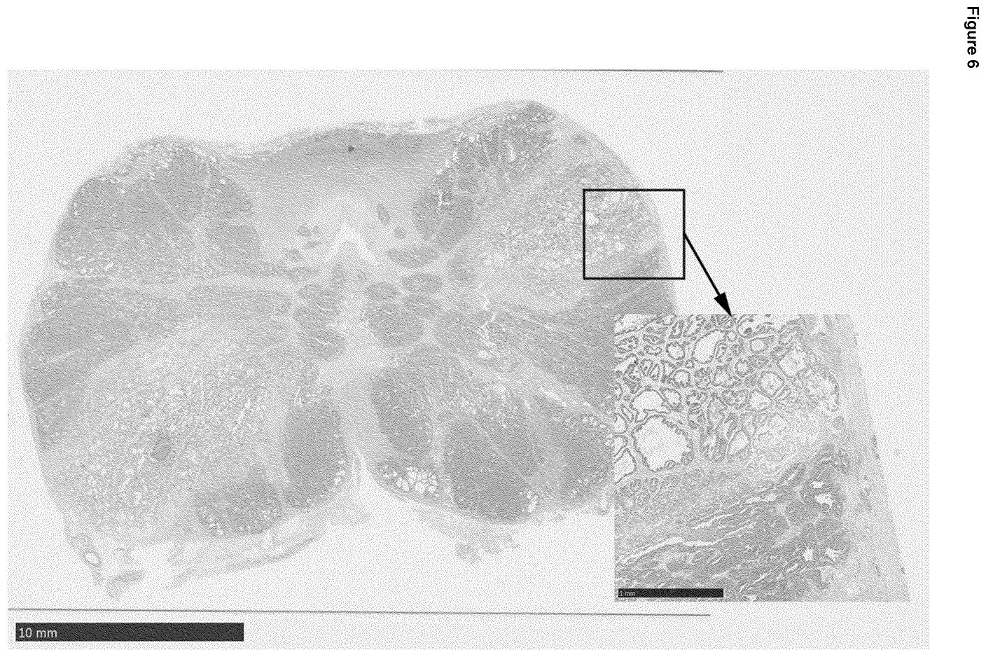

The average prostate volume of the experimental sample was 8875.48±1576.44 mm³. The area infiltrated by EtOH was predictable in size and shape, with a high-level correlation between the volume of EtOH injected and the volume of prostate tissue lesion. EtOH infusion proved impossible in one animal due to small prostate size. No extraprostatic leak during injection was noted in any of the remaining animals. The infusion pressure reached the maximum of 234±36 mm Hg and remained stable throughout the injection. On histological evaluation of the dose-finding experiment, a necrotic lesion was identified involving most of the prostate tissue and irregular in shape. The two lesion sites in this dog overlapped, likely due to the high volume injected, preventing establishing the correlation between the volume of ethanol and volume of the necrotic lesion (FIGS. 1 and 2). In the remainder of the prostates, EtOH diffused throughout the glandular tissue and smooth muscle, resulting in glandular dilatation, shrinkage of cell nuclei, and loss of visible nucleoli. EtOH volume of 0.5-2 ml caused well delineated necrotic lesions, which when reconstructed, were ellipsoid in shape and followed the needle track (FIG. 5). The pseudocapsule remained intact without evidence of disruption, even in cases when intraprostatic necrotic area developed adjacent to it. No EtOH effects on the structures surrounding the prostate were identified (FIG. 6). Injections of 0.5, 1.0, 1.5, and 2.0 ml with the 1 cm porous segment resulted in lesion volume 208±58, 588±112, 791±338, and 1158±200 mm³, respectively. The 2 cm porous segment needle resulted in larger areas of tissue infiltration. A regression analysis of the relationship between the volume of EtOH injected and the size of necrosis showed an R² of 0.85 (FIG. 2).

In general, for dogs, a linear correlation between the to be pre-determined administered volume of the composition and the volume of the (pre-determined) lesion may be determined by the following formula: y=0.612*x+0.075 (Formula), wherein y corresponds to the volume of said predetermined lesion, in ml, and x corresponds to the volume of said to be administered composition, in ml; with a regression analysis of R² being 0.8493.

### Chronic Animal Study

No extraprostatic leak during injection was noted in any of the remaining animals. The maximum infusion pressure was 221±47 mm Hg. All injections were free of exptraprostatic leak. The blood alcohol levels ranged between 0.5 to 4.3 mmol/l (Table 1). No immediate complications occurred. The long-term complications included swelling of the wound in two animals and serous exudation in one animal-no pain, hematuria, blood in stool or other symptoms were noted. The animals with swelling experienced a transient increase in body temperature to 38.5 and 39° C., which lasted less than 24 hours.

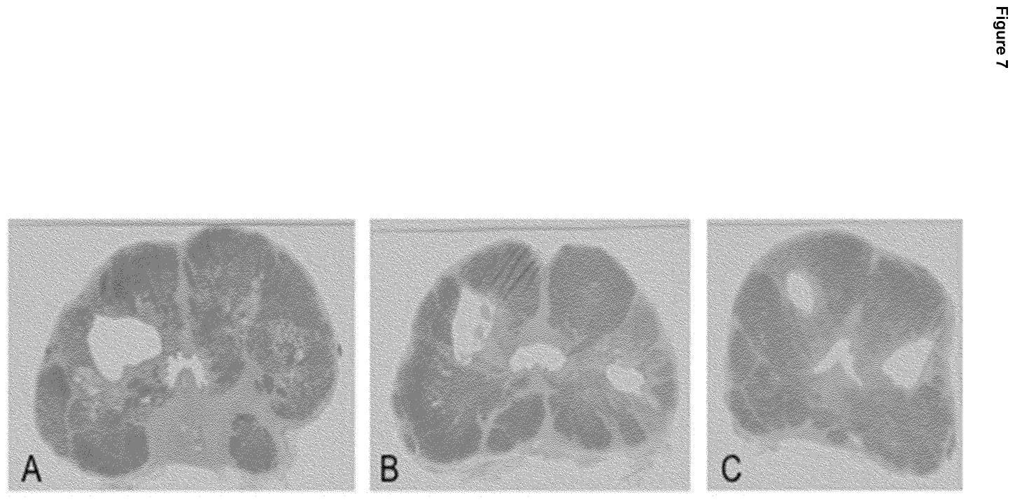

At the time of prostatectomy and euthanasia, examination revealed no damage to adjacent structures. No macroscopic abnormalities were visible on surface of the prostate. Hematoxylin and eosin stained whole mount sections of the prostate showed tissue defects (cavities) in all prostatic lobes injected with 1, 1.5, and 2 ml (FIG. 7). Tissue defects were lined with epithelium and surrounded by connective tissue-containing cells typical of early fibrosis. The area of the prostatic pseudocapsule, consisting of loose connective tissue, showed no abnormalities.

### Cadaveric Human Prostates

In general, for prostates from hearth beating cadaveric organ donors, a linear correlation between the to be pre-determined administered volume of the composition and the volume of the (pre-determined) lesion may be determined by the following formula: y=0.763*x−0.549 (Formula), wherein y corresponds to the volume of said predetermined lesion, in ml, and x corresponds to the volume of said to be administered composition in ml; with a regression analysis of R² being 0.4812.

It was observed that there is moderate correlation between the volume injected and the volume of the lesion and that injection of 3 ml created lesion on average is 1.78×bigger than that created with 2 ml. Similar to the findings obtained in canines, lesions in human prostates were regular and oval in shape. In a small number of instances the shape was irregular, most likely due to the portion of EtOH which in addition to diffuse convection type traveled through the lumen of the larger gland. In all cases the lesion was localized within the prostate. Prostatic capsule remained intact and no evidence to the extraprostatic tissue injury was observed.

### Items

1. A composition comprising an anti-prostate cancer agent for use in a method of focally treating prostate cancer in a human patient, said method comprising the step of transperineally, transrectally, or transurethrally administering through a hollow fiber membrane portion of a catheter a pre-determined volume of said composition which pervades essentially the entire volume of a pre-determined intraprostatic lesion.
2. The composition for the use of item 1, wherein the administration step is preceded by the step of transperineally, transrectally or transurethrally positioning the hollow fiber membrane portion of the catheter comprising said composition in the geometrical center of a pre-determined intraprostatic lesion, such that the middle of said hollow fiber membrane portion is in the geometrical center of the intraprostatic lesion.
3. The composition for the use of item 2, wherein MRI-TRUS fusion is applied for positioning the catheter comprising said composition in the geometrical center of said pre-determined intraprostatic lesion.
4. The composition for the use of any one of the preceding items, wherein the pre-determined volume of said composition is based on a linear correlation between the to be administered volume of said composition and the volume of said predetermined lesion.
5. The composition for the use of item 4, wherein the linear correlation is determined by the following formula:

$$y = {0.7634}^{*} x - 0.5488 , \qquad ( Formula I )$$

wherein y corresponds to the volume of said predetermined lesion, in ml, and x corresponds to the volume of said to be administered composition, in ml; with a regression analysis of R² being 0.4812.
6. The composition for the use of any one of the preceding items, wherein the pre-determined volume of said composition is calculated by the following formula:

$$x = ( y + 0.5488 ) / 0.7634 , \qquad ( Formula II )$$

wherein y corresponds to the volume of said predetermined lesion, in ml, and x corresponds to the pre-determined volume of said composition to be administered, in hml.
7. The composition for the use of any one of the preceding items, wherein said composition pervades essentially the entire volume of the pre-determined intraprostatic lesion, e.g. within 1 to 4 hours, preferably 1 to 3 hours, more preferably 1 to 2 hours after administration.
8. The composition for the use of any one of the preceding items, wherein the pre-determined volume of said composition is administered by the aid of a pump.
9. The composition for the use of any one of the preceding items, wherein the hollow fiber membrane of said catheter has the following characteristics
   wall thickness of 100+/−25 μm, and/or
   inner diameter 300+/−40 μm.
10. The composition for the use of any one of the preceding items, wherein the hollow fiber membrane of said catheter has the following membrane performance characteristics:
   maximum pore size 0.5+/−0.1 μm, and/or
   transmembrane flow (H₂O, 25° C.)≥35 ml/[min×cm²×bar].
11. The composition for the use of any one of the preceding items, wherein the hollow fiber membrane portion has a length of 0.5 to 5 cm, preferably 1 to 3 cm.
12. The composition for the use of any one of the preceding items, wherein the catheter is comprised by a catheter deployment system.
13. The composition for the use of any one of the preceding items, wherein said anti-prostate cancer agent is ethanol.
14. A method of focally treating prostate cancer in a human patient, said method comprising transperineally, transrectally, or transurethrally administering a pre-determined volume of a composition comprising an anti-prostate cancer agent through a hollow fiber membrane of a catheter to said patient in need thereof, wherein said composition pervades essentially the entire volume of a pre-determined intraprostatic lesion.

1. A a method of focally treating prostate cancer in a human patient, said method comprising: transperineally, transrectally, or transurethrally administering through a hollow fiber membrane portion of a catheter a pre-determined volume of an anti-prostate cancer agent to pervade essentially the entire volume of a pre-determined intraprostatic lesion, wherein the pre-determined volume of said composition is determined based on a linear correlation between the to be administered volume of said composition and the volume of said predetermined lesion.

2. The method of claim 1, wherein the administration step is preceded by the step of transperineally, transrectally or transurethrally positioning the hollow fiber membrane portion of the catheter comprising said composition in the geometrical center of a pre-determined intraprostatic lesion, such that the middle of said hollow fiber membrane portion is in the geometrical center of the intraprostatic lesion.

3. The use method of claim 2, wherein MRI-TRUS fusion is applied for positioning the catheter comprising said composition in the geometrical center of said pre-determined intraprostatic lesion.

4. The method of claim 1, wherein the linear correlation is determined by the following formula:
$$y = 0.7634 * x - 0.5488 , \qquad ( Formula I )$$
wherein y corresponds to the volume of said predetermined lesion, in ml, and x corresponds to the volume of said to be administered composition, in ml; with a regression analysis of R² being 0.4812.

5. The method of claim 1, wherein the pre-determined volume of said composition is calculated by the following formula:
$$x = ( y + 0.5488 ) / 0.7634 , \qquad ( Formula II )$$
wherein y corresponds to the volume of said predetermined lesion, in ml, and x corresponds to the pre-determined volume of said composition to be administered, in hml.

6. The method of claim 1, wherein said composition pervades essentially the entire volume of the pre-determined intraprostatic lesion, e.g. within 1 to 4 hours, preferably 1 to 3 hours, more preferably 1 to 2 hours after administration.

7. The method of claim 1, wherein the pre-determined volume of said composition is administered by the aid of a pump.

8. The method of claim 1, wherein the hollow fiber membrane of said catheter has the following characteristics
wall thickness of 100+/−25 μm, and/or
inner diameter 300+/−40 μm.

9. The method of claim 1, wherein the hollow fiber membrane of said catheter has the following membrane performance characteristics:
maximum pore size 0.5+/−0.1 μm, and/or
transmembrane flow (H₂O, 25° C.)≥35 ml/[min×cm²×bar].

10. The method of claim 1, wherein the hollow fiber membrane portion has a length of 0.5 to 5 cm, preferably 1 to 3 cm.

11. The method of claim 1, wherein the catheter is comprised by a catheter deployment system.

12. The method of claim 1, wherein said anti-prostate cancer agent is ethanol.

13. (canceled)

14. The method of claim 1, wherein said anti-prostate cancer agent is 2-hydroxyflutamide.

15. The method of claim 1, wherein said anti-prostate cancer agent pervades the entire volume of the pre-determined intraprostatic lesion within 1 to 4 hours.
